(19) United States
(12) Patent Application Publication
(10) Pub. No.: US 2012/0271263 A1
(43) Pub. Date: Oct. 25, 2012

(54) Penile urine absorber

(76) Inventor: Stanfield Laranard Hunter, Abilene, TX (US)

(21) Appl. No.: 13/066,569

(22) Filed: Apr. 19, 2011

(51) Int. Cl. A61F 13/45

(52) U.S. Cl. 604/377

(57) ABSTRACT

A penile urine absorber is disclosed, comprised of an outer vinyl casing, an inner cotton mat lining, and elastic ring band used for securely holding penile urine absorber.

The penile urine absorber may be fabricated in a variety of size and colors that is economical to manufacture, thereby providing a compact penile urine absorber to keep your penile body area clean, and penile clothes area dry.

A penile urine absorber comes assembled and ready to use.

### BACKGROUND OF THE INVENTION

This invention relates to a penile urine absorber.

The penile leak urine after urination.

Penile urine absorber of the type contemplated by this invention will fold compact flat when not in use.
Thus will be easy to store on person, and convenient to carry in pocket such as for traveling and social settings.

Applicant knows of no other patents granted to penile urine absorber.

### OBJECTS OF THE INVENTION

Accordingly, it is an object of the present invention to provide a penile urine absorber which prevents urine leak on penile body area and clothes.

It is a further object of the present invention to provide a penile urine absorber manufactured from materials which absorb urine and keep wetness from penile body area.

It is a still further object of the present invention to provide a penile urine absorber.

Another object of the present invention is to provide a penile urine absorber to keep body penile area dry.

It is still another object of the present invention to provide a penile urine absorber of various size.

It is still another object of the invention to provide a penile urine absorber in various colors.

Yet another object of the present invention is to provide a penile urine absorber for personal use.

A further object of the present invention is to provide a penile urine absorber that is ready to use.

Other objects and advantage of the present invention will become apparent by reading the detail description of the invention in conjunction with the accompanying drawings, in which:

### BRIEF DESCRIPTION OF THE DRAWINGS

FIG. 1 illustrates a plan view of a work for a penile urine absorber comprising one vinyl outer casing, one cotton inner mat lining, and one elastic ring band.

FIG. 1A is a view of a penile urine absorber showing a vinyl outer casing.

FIG. 2 is a view of a penile urine absorber showing a cotton inner mat lining.

FIG. 3 is a view of a penile urine absorber showing a elastic ring band.

### DETAILED DESCRIPTION OF THE PREFERRED EMBODIMENT

Those familiar in the art will readily see and understand the simplicity of the present invention, from the schedule one vinyl outer casing, one cotton inner mat lining, and one elastic ring band that comprise the penile urine absorber.
Therefore, it is understood that the penile urine absorber could be modified to accommodate any size.
The penile urine absorber itself is manufactured in various sizes to accommodate personal use.
It could be custom fabricated easily to accommodate almost any size need.
For the purpose of identifying the different parts of the present invention, please refer to the “Component List” page included in this application.

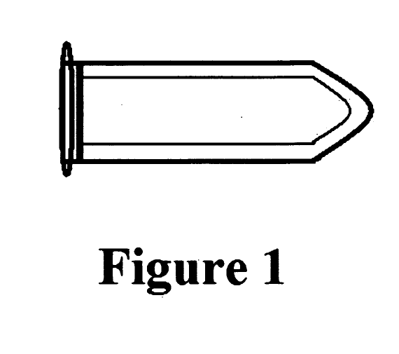

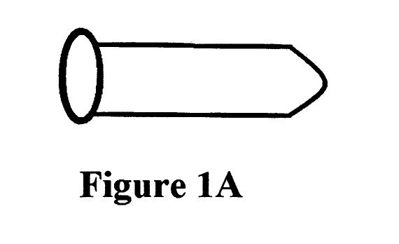

Referring to the drawing in FIG. 1, illustrates a schedule one vinyl outer casing, one cotton inner mat lining, and one elastic ring band assembled to make a penile urine absorber in accordance with the present invention, which is designed to absorb penile urine leak.

For the purpose of clarity and explanation, the following description and explanation of a penile urine absorber will be for a penile urine absorber that keep penile body area clean, and penile clothes area dry from penile urine leak after urination, approximately two inches by seven inches by one-fourth inch thick. Drawing FIG. 1, in order to build and assembled a penile urine absorber as illustrated, the following components will be used:

Schedule one vinyl outer casing, one cotton inner mat lining casing, and one elastic ring band;

(1) schedule vinyl outer casing approximately two inches by seven inches by one-fourth inch thick shown FIG. 1,

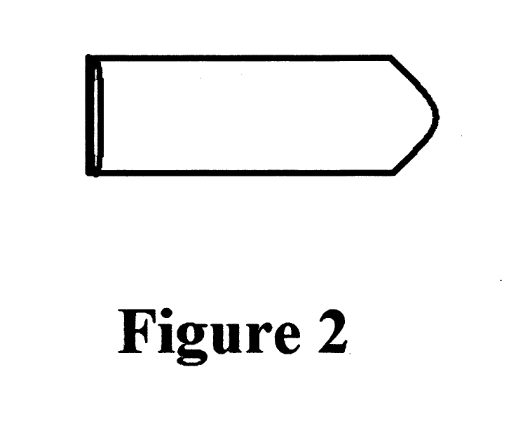

(1) schedule cotton inner mat lining casing approximately two inches by seven inches by one-fourth inch thick shown FIG. 2;

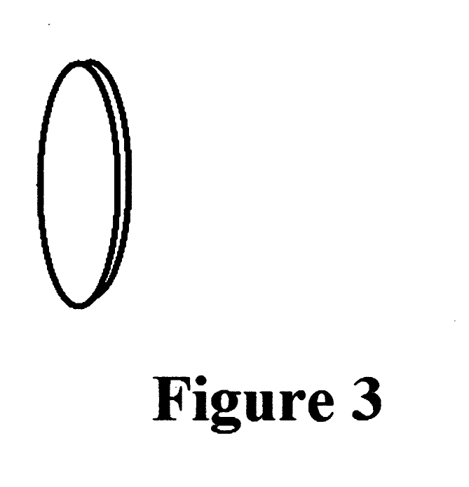

(1) schedule elastic ring band one inch, elastically two inch diameter by one fourth inch width shown FIG. 3;

Various changes and modifications may be made to the penile urine absorber without departing from the spirit or scope of the invention, and the above description is meant to be illustrative only.

1. A penile urine absorber comprising:
a. A schedule (1) vinyl outer casing penile sleeve;
b. A schedule (1) cotton inner mat lining
c. A schedule (1) elastic ring band used for securely holding penile urine absorber

2. A penile urine absorber as define in claim one, wherein said schedule (1) vinyl outer casing penile sleeve
(1) cotton inner mat lining and (1) elastic band is adjustable in order to secure any size penile urine absorber for personal use.
